(19) United States
(12) Patent Application Publication
(10) Pub. No.: US 2019/0263765 A1
(43) Pub. Date: Aug. 29, 2019

(54) SYNERGISTIC ANTICOAGULANT COMPOSITION

(71) Applicant: WISYS TECHNOLOGY FOUNDATION, INC., Madison, WI (US)

(72) Inventor: David E. Lewis, Eau Claire, WI (US)

(21) Appl. No.: 16/282,941

(22) Filed: Feb. 22, 2019

Related U.S. Application Data
(60) Provisional application No. 62/634,304, filed on Feb. 23, 2018.

(51) Int. Cl. C07D 303/06; C07D 303/31; A61K 31/37; A61P 7/02

(52) U.S. Cl. CPC C07D 303/06; A61P 7/02; A61K 31/37; C07D 303/31

(57) ABSTRACT

A composition is provided that, when utilized in combination with a coumarin anticoagulant, greatly improves the anticoagulant effects of the anticoagulant in mammalian subjects. The composition is a compound having a naphthohydroquinone ring system substantially similar to the ring system of the reduced form of vitamin K₁ and has the general formula:

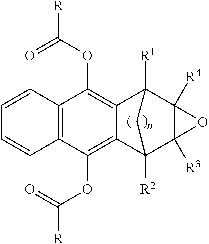

where n is 1 or 2, where R₁ and R₄ are hydrogen or acyl, R₂ is a hydrogen or saturated or unsaturated alkyl group with up to 6 carbons, and R₃ is a hydrogen or saturated or unsaturated alkyl group with up to 6 carbons, or R₂ and R₃ are part of a cyclic or polycyclic ring system.

### CROSS-REFERENCE TO RELATED APPLICATIONS

This application claims the benefit of U.S. Provisional Application No. 62/634,304, filed Feb. 23, 2018, and hereby incorporated by reference.

### FIELD OF THE INVENTION

A new class of compounds has been developed that exhibits cooperative activity with warfarin to quadruple the anticoagulant activity of the coumarin, while not exhibiting significant anticoagulant activity when administered alone.

### BACKGROUND OF THE INVENTION

Various coumarin anticoagulant compositions, and in particular, warfarin, have long been utilized in different human and animal modalities, e.g., for the treatment of atrial fibrillation in humans, and as the active principle of rodenticide baits. However, as a result of the continued use of the compound warfarin as a poison for, in particular, rats and mice, rodents have become less susceptible to the effects of warfarin. In recent years, this resistance to coumarin anticoagulants has also begun to emerge with the second-generation coumarin anticoagulants such as brodifacoum.

The coumarin anticoagulants act by inhibiting the enzyme VKORC1 (Vitamin K Oxidoreductase Complex 1), which is the only enzyme in mammals that is capable of reducing the oxidized form of vitamin K, vitamin K epoxide, to vitamin K itself VKORC1 is therefore a susceptible bottleneck in the vitamin K cycle (FIG. 1).

By inhibiting this enzyme, these anticoagulants prevent the recycling of vitamin K, and they thus slow the production of mature clotting factors (e.g., thrombin) from their precursors (e.g, prothrombin) by reducing the total amount of the active form of vitamin K available to the animal. The inhibition of VKORC1 by coumarin anticoagulants is competitive because the anticoagulation can be reversed by high doses of vitamin K. However, the effectiveness of the vitamin K rescue mechanism depends markedly on the coumarin anticoagulant itself: warfarin is cleared from the body in approximately 72-96 hours, while the clearance of (the much more lipophilic) brodifacoum requires months, necessitating long-term treatment with vitamin K to prevent death.

Therefore, it is desirable to develop a composition that can be utilized either as a substitute or as a complement to existing anticoagulant compositions, such as warfarin, to increase the effectiveness of the anticoagulant. It is also desirable to develop a composition that will decompose to inactive compounds following the death of the rodent, or that has a relatively short half-life to permit rescue of accidental intoxication of non-target animals (e.g., fish, family pets, raptors and scavengers, small children). It is also desirable to develop a new composition that will act by a mechanism other than the competitive inhibition of VKORC1 so that this mechanism of resistance to coumarin anticoagulants may be circumvented. One possible alternative target for inhibition is the cytochrome P₄₅₀, polymorph CYP 2C9, which oxidized warfarin to the inactive 7-hydroxywarfarin. By inhibiting this enzyme, the oxidation of warfarin to inactive 7-hydroxywarfarin may be slowed or completely stopped, resulting in a net increase in the circulating concentration in the blood at the same oral dosage (FIG. 2).

Our studies to date have elucidated the probable course of the initial metabolism of these compounds in the stomach and duodenum of the animal. UWEC-K2, the active principle when co-administered with warfarin, is metabolized to a diol under acidic conditions (0.1 M HCl in 50% aqueous acetone, mimicking stomach acid) and to a cyclopropyl ketone on brief exposure to base (potassium carbonate in methanol, mimicking the duodenum). These outcomes are summarized in FIG. 3.

Extensive experiments with UWEC-K2 and warfarin under a wide range of conditions has not yielded significant amounts of the conjugate proposed previously. See Stromich, J. J.; Weber, A. K.; Mirzaei, Y. R.; Caldwell, M. D.; Lewis, D. E. Bioorg. Med. Chem. Lett. 2010, 20, 1928-1932, hereby incorporated by reference. Thus, the dominant pathway of action may not be as previously proposed.

### SUMMARY OF THE INVENTION

According to one aspect of the present invention, a composition has been developed that, when utilized in combination with warfarin or 7-hydroxywarfarin, acts to greatly improve the anticoagulant effects of the coumarin in mammalian subjects.

The composition includes a compound or mixture of compounds obtained by hydrolysis of an epoxy-substituted 1,4-naphthalenediol diester derived from a Diels-Alder adduct of 1,4-naphthoquinone and a substituted cyclopentadiene or 1,3-cyclohexadiene, and having general structures I and II:

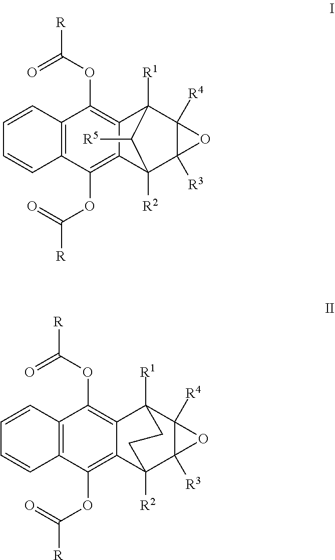

where R is a hydrogen or saturated or unsaturated alkyl group with up to 6 carbons; and where R¹ , R² , R³, R⁴ and R⁵ are hydrogen, alkyl, alkoxy, trialkylsilyl or trialkylsilyloxy; and

R¹ is a hydrogen or a saturated or unsaturated alkyl group with up to 6 carbons;
or an alkoxy group with up to 3 carbons; or
a trialkylsilyl group (trimethylsilyl, triethylsilyl, tent-butyldimethylsilyl, dimethylphenylsilyl); or
a trialkylsilyloxy group (trimethylsilyloxy, tent-butyldimethylsilyloxy, triethylsilyloxy, dimethylphenylsilyloxy); and

R² is a hydrogen or a saturated or unsaturated alkyl group with up to 6 carbons;
or an alkoxy group with up to 3 carbons; or
a trialkylsilyl group (trimethylsilyl, triethylsilyl, tent-butyldimethylsilyl, dimethylphenylsilyl); or
a trialkylsilyloxy group (trimethylsilyloxy, tent-butyldimethylsilyloxy, triethylsilyloxy, dimethylphenylsilyloxy); and

R³ is a hydrogen or a saturated or unsaturated alkyl group with up to 6 carbons; or
or an alkoxy group with up to 3 carbons; or
a trialkylsilyl group (trimethylsilyl, triethylsilyl, tent-butyldimethylsilyl, dimethylphenylsilyl); or
a trialkylsilyloxy group (trimethylsilyloxy, tent-butyldimethylsilyloxy, triethylsilyloxy, dimethylphenylsilyloxy); and

R⁴ is a hydrogen or a saturated or unsaturated alkyl group with up to 6 carbons; or
or an alkoxy group with up to 3 carbons; or
a trialkylsilyl group (trimethylsilyl, triethylsilyl, tert-butyldimethylsilyl, dimethylphenylsilyl); or
a trialkylsilyloxy group (trimethylsilyloxy, tent-butyldimethylsilyloxy, triethylsilyloxy, dimethylphenylsilyloxy); and

R⁵ is a hydrogen or a trialkylsilyl group (trimethylsilyl, triethylsilyl, tert-butyldimethylsilyl, dimethylphenylsilyl);

or

where R¹ and R⁴ comprise a substituted or unsubstituted, saturated or unsaturated α,ω-alkylene group with up to four carbons; and

R² is a hydrogen or saturated or unsaturated alkyl group with up to 6 carbons;
or an alkoxy group with up to 3 carbons; or
a trialkylsilyl group (trimethylsilyl, triethylsilyl, tert-butyldimethylsilyl, dimethylphenylsilyl); or
a trialkylsilyloxy group (trimethylsilyloxy, tent-butyldimethylsilyloxy, triethylsilyloxy, dimethylphenylsilyloxy); and

R³ is a hydrogen or saturated or unsaturated alkyl group with up to 6 carbons; or
or an alkoxy group with up to 3 carbons; or
a trialkylsilyl group (trimethylsilyl, triethylsilyl, tert-butyldimethylsilyl, dimethylphenylsilyl); or
a trialkylsilyloxy group (trimethylsilyloxy, tent-butyldimethylsilyloxy, triethylsilyloxy, dimethylphenylsilyloxy); and

R⁵ is a hydrogen or a trialkylsilyl group (trimethylsilyl, triethylsilyl, tert-butyldimethylsilyl, dimethylphenylsilyl);

or

where R³ and R⁴ comprise a substituted or unsubstituted, saturated or unsaturated α,ω-alkylene group with up to four carbons; and

R¹ is a hydrogen or a saturated or unsaturated alkyl group with up to 6 carbons;
or an alkoxy group with up to 3 carbons; or
a trialkylsilyl group (trimethylsilyl, triethylsilyl, tent-butyldimethylsilyl, dimethylphenylsilyl); or
a trialkylsilyloxy group (trimethylsilyloxy, tent-butyldimethylsilyloxy, triethylsilyloxy, dimethylphenylsilyloxy); and

R² is a hydrogen or a saturated or unsaturated alkyl group with up to 6 carbons;
or an alkoxy group with up to 3 carbons; or
a trialkylsilyl group (trimethylsilyl, triethylsilyl, tent-butyldimethylsilyl, dimethylphenylsilyl); or
a trialkylsilyloxy group (trimethylsilyloxy, tent-butyldimethylsilyloxy, triethylsilyloxy, dimethylphenylsilyloxy); and

R⁵ is hydrogen or a trialkylsilyl group (trimethylsilyl, triethylsilyl, tert-butyldimethylsilyl, dimethylphenylsilyl).

The hydrolysis mixture contains as a major component, a compound based on the 4,5-benzotricyclo[6.2.1.0^{2,10}]undec-4-en-3,6-dione (III) or the 4,5-benzotricyclo[6.2.2.0^{2,11}]-dodec-4-en-3,6-dione (IV):

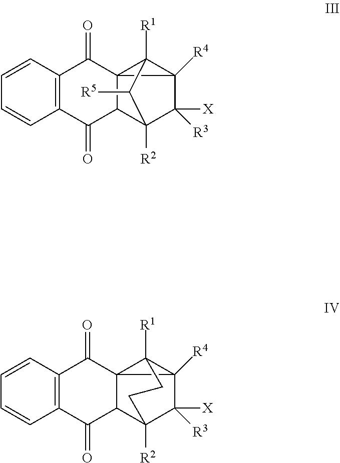

where R¹, R² , R³ and R⁴ are hydrogen, alkyl, alkoxy, silyl or silyloxy; and

R¹ is a hydrogen or saturated or unsaturated alkyl group with up to 6 carbons;
or an alkoxy group with up to 3 carbons; or
a trialkylsilyl group (trimethylsilyl, triethylsilyl, tent-butyldimethylsilyl, dimethylphenylsilyl); or
a trialkylsilyloxy group (trimethylsilyloxy, tent-butyldimethylsilyloxy, triethylsilyloxy, dimethylphenylsilyloxy); and

R² is a hydrogen or saturated or unsaturated alkyl group with up to 6 carbons;
or an alkoxy group with up to 3 carbons; or
a trialkylsilyl group (trimethylsilyl, triethylsilyl, tent-butyldimethylsilyl, dimethylphenylsilyl); or
a trialkylsilyloxy group (trimethylsilyloxy, tent-butyldimethylsilyloxy, triethylsilyloxy, dimethylphenylsilyloxy); and

R³ is a hydrogen or saturated or unsaturated alkyl group with up to 20 carbons; or
or an alkoxy group with up to 3 carbons; or
a trialkylsilyl group (trimethylsilyl, triethylsilyl, tent-butyldimethylsilyl, dimethylphenylsilyl); or
a trialkylsilyloxy group (trimethylsilyloxy, tent-butyldimethylsilyloxy, triethylsilyloxy, dimethylphenylsilyloxy); and

R⁴ is a hydrogen or saturated or unsaturated alkyl group with up to 20 carbons; or
or an alkoxy group with up to 3 carbons; or
a trialkylsilyl group (trimethylsilyl, triethylsilyl, tent-butyldimethylsilyl, dimethylphenylsilyl); or
a trialkylsilyloxy group (trimethylsilyloxy, tent-butyldimethylsilyloxy, triethylsilyloxy, dimethylphenylsilyloxy); or

R¹ and R⁴ are part of a cyclic or polycyclic ring system; or

R³ and R⁴ are part of a cyclic or polycyclic ring system.

and where X is a heteratom group chosen from:

an alcohol;

an ether, OR⁴, where R⁴ is a hydrogen or saturated or unsaturated alkyl group with up to 6 carbons;

an ester of a carboxylic acid with up to six carbon atoms;

a halide (bromide, chloride or iodide);

a sulfonate ester (methanesulfonate, benzenesulfonate, p-toluenesulfonate, p-bromobenzenesulfonate, p-nitrobenzenesulfonate, naphthalene-1-sulfonate, naphthalene-2-sulfonate);

an amine, NR⁵R⁶, where R⁵ and R⁶ are hydrogen or an alkyl group with up to 6 carbons;

and a coumarin-based anticoagulant or metabolite thereof with the general structure

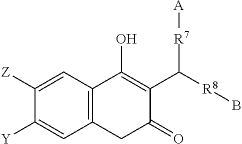

where R⁷ is a substituted alkyl group with up to 6 carbons; and

A is a group chosen from an alcohol, an ester, an ether, an ester, or a ketone; and

where R⁸ is a substituted alkyl group with up to 6 carbons; and

B is a an aryl group (phenyl, biphenyl, furyl, 4′-bromo-4,1′-biphenyl);

or where R⁷ and R⁸ constitute a cycloalkyl group with up to 7 carbons; and

A is a group chosen from an alcohol, an ester, an ether, an ester, or a ketone; and

B is a an aryl group (phenyl, biphenyl, furyl, 4′-bromo-4,1′-biphenyl); and

where X is hydrogen or hydroxy; and
where Y is hydrogen or hydroxy.

### BRIEF DESCRIPTION OF THE DRAWINGS

FIG. 1 depicts a general schematic illustrating the vitamin K cycle;

FIG. 2 depicts a general schematic illustrating oxygenation of warfarin to 7-hydroxywarfarin by cytochrome P450 polymorph CYP 2C9;

FIG. 3 depicts a general schematic illustrating reactions of UWEC-K2 under conditions of acid and base hydrolysis;

FIG. 4 depicts a general schematic illustrating a putative model that rationalizes the reversal of the anticoagulant activity of warfarin in the presence of UWEC-K2;

FIG. 5 depicts a general schematic illustrating generation of a naphthoquinone-based free radical by oxidation of the cyclopropyl ketone;

FIG. 6 depicts a general schematic illustrating possible coupling of a free radical derived from a cyclo-propyl ketone with 7-hydroxywarfarin; and

FIG. 7 depicts a general schematic illustrating possible inhibition of CYP 2C9 by a conjugate of 7-hydroxywarfarin formed in situ.

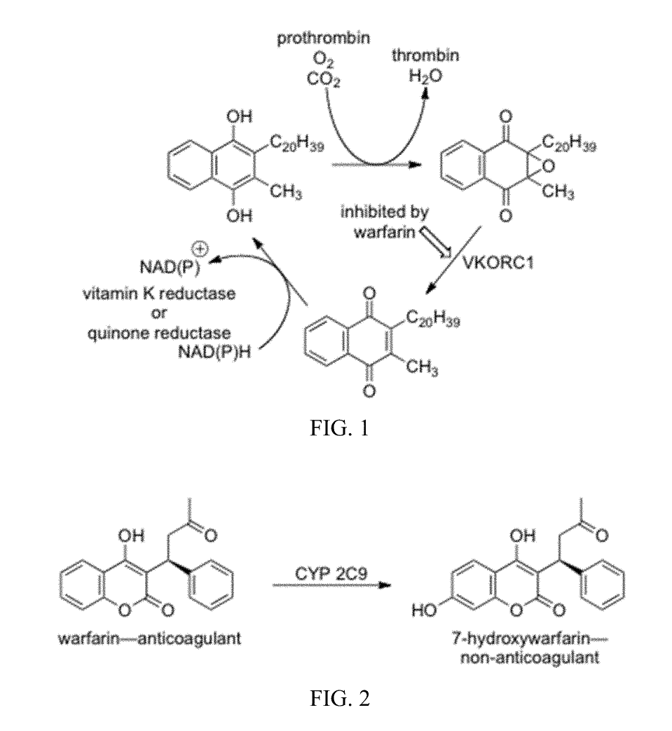

### DETAILED DESCRIPTION OF THE INVENTION

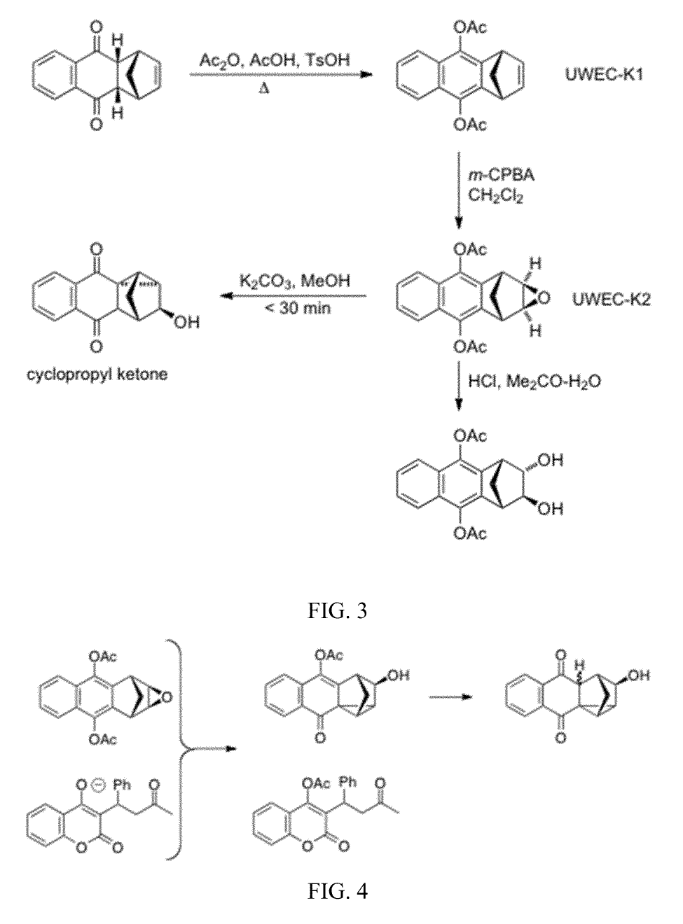

The invention consists of a two-component synergistic mixture of a cyclopropyl ketone and a coumarin anticoagulant or its metabolite. The compound designated UWEC-K2 is prepared from the Diels-Alder adduct of 1,4-naphthoquinone and cyclopentadiene by the method of Lewis, et al. See Lewis, D. E.; Caldwell, M. D. “Synergistic anticoagulant composition.” U.S. Pat. No. 8,765,982 B2 (Jul. 1, 2014), hereby incorporated by reference. The treatment of UWEC-K2 with potassium carbonate in methanol for a short period (not more than 30 minutes) results in the formation of the cyclopropyl ketone. The cyclopropyl ketone in FIG. 3 has been derived from the starting diketone by a somewhat different route. See Marchand, A. P.; Dong, E. Z.; Bott, S. G. “Synthesis and acid- and base-promoted ring opening of polycarbocyclic oxiranes.” Tetrahedron 1998, 54(18), 4459-4570, hereby incorporated by reference. The approach described herein does not require the use of the strong bases or extended reaction times required by the March and method, and does not require chromatography.

The epoxide is essential for UWEC-K2 activity. In one model, it is postulated that the synergistic anticoagulant activity involves the transesterification of warfarin sodium by UWEC-K2. This process will lead to ring opening of the epoxide to give the cyclopropyl ketone, which makes the otherwise unfavorable equilibrium for the acetyl transfer irreversible (UWEC-K1 does not exhibit this type of biological activity). It is postulated that the products of the transesterification (warfarin acetate and the cyclopropyl ketone) are not anticoagulants, which then provides a reasonable rationalization of the observation that the anticoagulant activity of warfarin (and the anticoagulant activity of UWEC-K1) is reversed (FIG. 4).

It follows from this that the hydrolysis of the warfarin acetate at a later time may lead to the generation of a bolus of warfarin that leads to the much higher levels of anticoagulation observed previously. See Stromich, J. J.; Weber, A. K.; Mirzaei, Y. R. ; Caldwell, M. D.; Lewis, D. E. Bioorg. Med. Chem. Lett. 2010, 20, 1928-1932, hereby incorporated by reference. Warfarin acetate is also more lipophilic than warfarin itself, which may increase its half-life in the animal, also rendering it a more potent anticoagulant.

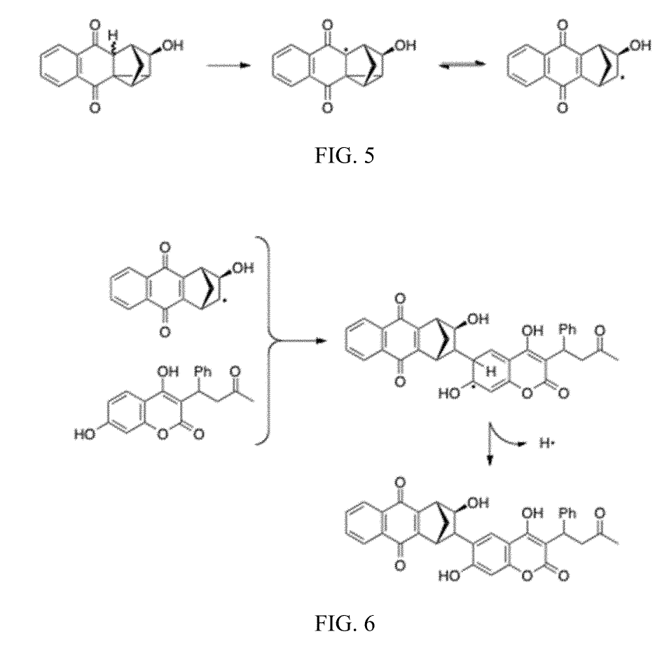

Another model that rationalizes the observed synergistic anticoagulant activity involves an intriguing possibility based on the typical activity of cytochrome P₄₅₀. In particular, if the cyclopropyl ketone is bound in the active site of the cytochrome, then CYP 2C9 could oxidize this to a free radical that undergoes the normal cyclopropylcarbinyl to homoallyl radical isomerization to generate a naphthoquinone and a simple alkyl radical (FIG. 5).

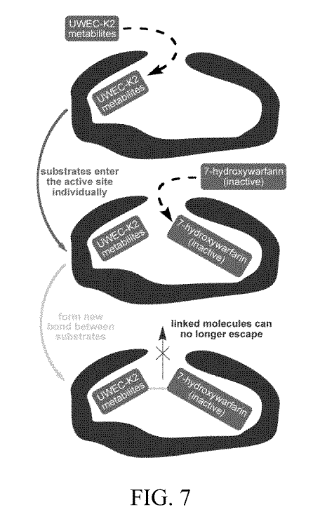

What is particularly enticing is the fact that this free radical could easily react with warfarin or, much more interestingly, 7-hydroxywarfarin, to give a coupled product that may well be a super inhibitor of CYP 2C9 (FIG. 6).

The available evidence about this enzyme is that it has a large active site that can exist on an “open” form and a “closed” form. The X-ray crystal structures show that both active sites can accommodate a second molecule in addition to the warfarin. We suggest that the opening to the active site, however, is such that only one molecule at a time may enter, and that once the UWEC-K2 metabolite and the 7-hydroxywarfarin enter the active site of the enzyme, they actually become covalently bonded to each other. This results in a molecule that is too big to exit the active site, and thus leads to inactivation of the enzyme (FIG. 7). Since CYP 2C9 provides the most important pathway for deactivating warfarin in vivo, this may provide a new way for the control of warfarin anticoagulation.

The initial reversal of warfarin anticoagulation by UWEC-K2 may be rationalized in terms of the cyclopropyl ketone binding to the active site of CYP 2C9, and making warfarin and 7-hydroxywarfarin bind much more tightly, thus reducing the available warfarin in circulation, making the anticoagulation by warfarin ineffective. Once the CYP 2C9 active site is saturated, however, the effective warfarin concentration will rise, and anticoagulation will increase.

Alternatively, the final conjugate, formed in the active site of CYP 2C9 by coupling of warfarin (or 7-hydroxywarfarin) with the naphthoquinone radical may be a super inhibitor of CYP 2C9. This should gradually lead to a dramatic rise in warfarin anticoagulation because the inactivation of warfarin by CYP 2C9 is prevented as the amount of uninhibited CYP 2C9 decreases. One would assume that there is some threshold level of inhibition required to see the increased effectiveness of warfarin as an anticoagulant.

One especially puzzling result of early in vivo testing of UWEC-K2 was the observation that when the diets were mixed using a small amount of alcohol to add the UWEC-K2 to the soybean oil, the resultant tests were negative for the anticoagulation enhancement. It is difficult to see how so small a change could have such a large effect, but we now believe that the ethanol may have activated esterases to cleave more of the UWEC-K2 in the stomach, with the result that less of the UWEC-K2 entered the duodenum unaltered. Under these conditions, less UWEC-K2 would also enter the liver, where it is metabolized to the active adjuvant.

As can be appreciated, the results described in the above examples support the utility of the composition described and claimed herein that, when utilized in combination with a coumarin anticoagulant, improves the anticoagulant effects of the anticoagulant in mammalian subjects. Other embodiments and uses of the invention will be apparent to those skilled in the art from consideration from the specification and practice of the invention disclosed herein. All references cited herein for any reason, including all journal citations and U.S./foreign patents and patent applications, if present, are specifically and entirely incorporated herein by reference. It is understood that the invention is not confined to the specific compositions, materials, methods, formulations, manufacturing conditions, etc., herein illustrated and described, but embraces such modified forms thereof as come within the scope of the following claims.

What is claimed is:

1. A compound comprising the following structure: 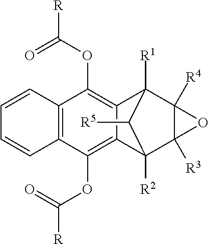
wherein R is hydrogen or saturated or unsaturated alkyl with up to 6 carbons; and
wherein R¹ is hydrogen or saturated or unsaturated alkyl with up to 6 carbons; or alkoxy with up to 3 carbons; or trialkylsilyl (trimethylsilyl, triethylsilyl, tert-butyldimethylsilyl, dimethylphenylsilyl); or trialkylsilyloxy (trimethylsilyloxy, tert-butyldimethylsilyloxy, triethylsilyloxy, dimethylphenylsilyloxy); and
wherein R² is hydrogen or saturated or unsaturated alkyl with up to 6 carbons; or alkoxy with up to 3 carbons; or trialkylsilyl (trimethylsilyl, triethylsilyl, tert-butyldimethylsilyl, dimethylphenylsilyl); or rialkylsilyloxy (trimethylsilyloxy, tert-butyldimethylsilyloxy, triethylsilyloxy, dimethylphenylsilyloxy); and
R³ is hydrogen or saturated or unsaturated alkyl with up to 6 carbons; or alkoxy with up to 3 carbons; or trialkylsilyl (trimethylsilyl, triethylsilyl, tert-butyldimethylsilyl, dimethylphenylsilyl); or trialkylsilyloxy (trimethylsilyloxy, tert-butyldimethylsilyloxy, triethylsilyloxy, dimethylphenylsilyloxy); and
R⁴ hydrogen or saturated or unsaturated alkyl with up to 6 carbons; or alkoxy with up to 3 carbons; or trialkylsilyl (trimethylsilyl, triethylsilyl, tert-butyldimethylsilyl, dimethylphenylsilyl); or trialkylsilyloxy (trimethylsilyloxy, tert-butyldimethylsilyloxy, triethylsilyloxy, dimethylphenylsilyloxy); and
R⁵ is hydrogen or trialkylsilyl (trimethylsilyl, triethylsilyl, tert-butyldimethylsilyl, dimethylphenylsilyl);
or
wherein R¹ and R² comprise a substituted or unsubstituted, saturated or unsaturated α,ω-alkylene group with up to four carbons; and
wherein R³ is hydrogen or saturated or unsaturated alkyl with up to 6 carbons; or alkoxy with up to 3 carbons; or trialkylsilyl (trimethylsilyl, triethylsilyl, tert-butyldimethylsilyl, dimethylphenylsilyl); or rialkylsilyloxy (trimethylsilyloxy, tert-butyldimethylsilyloxy, triethylsilyloxy, dimethylphenylsilyloxy); and
wherein R⁴ is hydrogen or saturated or unsaturated alkyl with up to 6 carbons; or alkoxy with up to 3 carbons; or trialkylsilyl (trimethylsilyl, triethylsilyl, tert-butyldimethylsilyl, dimethylphenylsilyl); or trialkylsilyloxy (trimethylsilyloxy, tert-butyldimethylsilyloxy, triethylsilyloxy, dimethylphenylsilyloxy); and
wherein R⁵ is hydrogen or trialkylsilyl (trimethylsilyl, triethylsilyl, tert-butyldimethylsilyl, dimethylphenylsilyl);
or
wherein R¹ and R⁴ comprise a substituted or unsubstituted, saturated or unsaturated α,ω-alkylene group with up to four carbons; and
wherein R² is hydrogen or saturated or unsaturated alkyl with up to 6 carbons; or alkoxy with up to 3 carbons; or trialkylsilyl (trimethylsilyl, triethylsilyl, tert-butyldimethylsilyl, dimethylphenylsilyl); or rialkylsilyloxy (trimethylsilyloxy, tert-butyldimethylsilyloxy, triethylsilyloxy, dimethylphenylsilyloxy); and
wherein R³ is hydrogen or saturated or unsaturated alkyl with up to 6 carbons; or alkoxy with up to 3 carbons; or trialkylsilyl (trimethylsilyl, triethylsilyl, tert-butyldimethylsilyl, dimethylphenylsilyl); or trialkylsilyloxy (trimethylsilyloxy, tert-butyldimethylsilyloxy, triethylsilyloxy, dimethylphenylsilyloxy); and
wherein R⁵ is hydrogen or trialkylsilyl (trimethylsilyl, triethylsilyl, tert-butyldimethylsilyl, dimethylphenylsilyl).

2. A compound comprising the following structure: 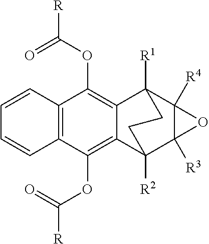
wherein R is hydrogen or saturated or unsaturated alkyl with up to 6 carbons; and
wherein R¹ is hydrogen or saturated or unsaturated alkyl with up to 6 carbons; or alkoxy with up to 3 carbons; or trialkylsilyl (trimethylsilyl, triethylsilyl, tert-butyldimethylsilyl, dimethylphenylsilyl); or trialkylsilyloxy (trimethylsilyloxy, tert-butyldimethylsilyloxy, triethylsilyloxy, dimethylphenylsilyloxy); and
wherein R² is hydrogen or saturated or unsaturated alkyl with up to 6 carbons; or alkoxy with up to 3 carbons; or trialkylsilyl (trimethylsilyl, triethylsilyl, tert-butyldimethylsilyl, dimethylphenylsilyl); or rialkylsilyloxy (trimethylsilyloxy, tert-butyldimethylsilyloxy, triethylsilyloxy, dimethylphenylsilyloxy); and
wherein R³ is hydrogen or saturated or unsaturated alkyl with up to 6 carbons; or alkoxy with up to 3 carbons; or trialkylsilyl (trimethylsilyl, triethylsilyl, tert-butyldimethylsilyl, dimethylphenylsilyl); or trialkylsilyloxy (trimethylsilyloxy, tert-butyldimethylsilyloxy, triethylsilyloxy, dimethylphenylsilyloxy); and
wherein R⁴ is hydrogen or saturated or unsaturated alkyl with up to 6 carbons; or alkoxy with up to 3 carbons; or trialkylsilyl (trimethylsilyl, triethylsilyl, tert-butyldimethylsilyl, dimethylphenylsilyl); or trialkylsilyloxy (trimethylsilyloxy, tert-butyldimethylsilyloxy, triethylsilyloxy, dimethylphenylsilyloxy);
or
wherein R¹ and R² comprise a substituted or unsubstituted, saturated or unsaturated α,ω-alkylene group with up to four carbons; and
wherein R³ is hydrogen or saturated or unsaturated alkyl with up to 6 carbons; or alkoxy with up to 3 carbons; or trialkylsilyl (trimethylsilyl, triethylsilyl, tert-butyldimethylsilyl, dimethylphenylsilyl); or rialkylsilyloxy (trimethylsilyloxy, tert-butyldimethylsilyloxy, triethylsilyloxy, dimethylphenylsilyloxy); and
wherein R⁴ is hydrogen or saturated or unsaturated alkyl with up to 6 carbons; or alkoxy with up to 3 carbons; or trialkylsilyl (trimethylsilyl, triethylsilyl, tert-butyldimethylsilyl, dimethylphenylsilyl); or trialkylsilyloxy (trimethylsilyloxy, tert-butyldimethylsilyloxy, triethylsilyloxy, dimethylphenylsilyloxy);
or
wherein R¹ and R⁴ comprise a substituted or unsubstituted, saturated or unsaturated α,ω-alkylene group with up to four carbons; and
wherein R² is hydrogen or saturated or unsaturated alkyl with up to 6 carbons; or alkoxy with up to 3 carbons; or trialkylsilyl (trimethylsilyl, triethylsilyl, tert-butyldimethylsilyl, dimethylphenylsilyl); or rialkylsilyloxy (trimethylsilyloxy, tert-butyldimethylsilyloxy, triethylsilyloxy, dimethylphenylsilyloxy); and
wherein R³ is hydrogen or saturated or unsaturated alkyl with up to 6 carbons; or alkoxy with up to 3 carbons; or trialkylsilyl (trimethylsilyl, triethylsilyl, tert-butyldimethylsilyl, dimethylphenylsilyl); or trialkylsilyloxy (trimethylsilyloxy, tert-butyldimethylsilyloxy, triethylsilyloxy, dimethylphenylsilyloxy).

3. A compound comprising the following structure: 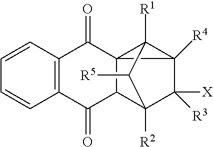
wherein R¹ is hydrogen or saturated or unsaturated alkyl with up to 6 carbons; or alkoxy with up to 3 carbons; or trialkylsilyl (trimethylsilyl, triethylsilyl, tert-butyldimethylsilyl, dimethylphenylsilyl); or trialkylsilyloxy (trimethylsilyloxy, tert-butyldimethylsilyloxy, triethylsilyloxy, dimethylphenylsilyloxy); and
wherein R² is hydrogen or saturated or unsaturated alkyl with up to 6 carbons; or alkoxy with up to 3 carbons; or trialkylsilyl (trimethylsilyl, triethylsilyl, tert-butyldimethylsilyl, dimethylphenylsilyl); or rialkylsilyloxy (trimethylsilyloxy, tert-butyldimethylsilyloxy, triethylsilyloxy, dimethylphenylsilyloxy); and
wherein R³ is hydrogen or saturated or unsaturated alkyl with up to 6 carbons; or alkoxy with up to 3 carbons; or trialkylsilyl (trimethylsilyl, triethylsilyl, tert-butyldimethylsilyl, dimethylphenylsilyl); or trialkylsilyloxy (trimethylsilyloxy, tert-butyldimethylsilyloxy, triethylsilyloxy, dimethylphenylsilyloxy); and
wherein R⁴ hydrogen or saturated or unsaturated alkyl with up to 6 carbons; or alkoxy with up to 3 carbons; or trialkylsilyl (trimethylsilyl, triethylsilyl, tert-butyldimethylsilyl, dimethylphenylsilyl); or trialkylsilyloxy (trimethylsilyloxy, tert-butyldimethylsilyloxy, triethylsilyloxy, dimethylphenylsilyloxy); and
wherein R⁵ is hydrogen or trialkylsilyl (trimethylsilyl, triethylsilyl, tert-butyldimethylsilyl, dimethylphenylsilyl); and
wherein X is a heteroatomic group (alcohol, ester, amine, amide, sulfonamide, halide, sulfonate ester);
or
wherein R¹ and R² comprise a substituted or unsubstituted, saturated or unsaturated α,ω-alkylene group with up to four carbons; and
wherein R³ is hydrogen or saturated or unsaturated alkyl with up to 6 carbons; or alkoxy with up to 3 carbons; or trialkylsilyl (trimethylsilyl, triethylsilyl, tert-butyldimethylsilyl, dimethylphenylsilyl); or rialkylsilyloxy (trimethylsilyloxy, tert-butyldimethylsilyloxy, triethylsilyloxy, dimethylphenylsilyloxy); and
wherein R⁴ is hydrogen or saturated or unsaturated alkyl with up to 6 carbons; or alkoxy with up to 3 carbons; or trialkylsilyl (trimethylsilyl, triethylsilyl, tert-butyldimethylsilyl, dimethylphenylsilyl); or trialkylsilyloxy (trimethylsilyloxy, tert-butyldimethylsilyloxy, triethylsilyloxy, dimethylphenylsilyloxy); and
wherein R⁵ is hydrogen or trialkylsilyl (trimethylsilyl, triethylsilyl, tert-butyldimethylsilyl, dimethylphenylsilyl); and
wherein X is a heteroatomic group (alcohol, ester, amine, amide, sulfonamide, halide, sulfonate ester);
or
wherein R¹ and R⁴ comprise a substituted or unsubstituted, saturated or unsaturated α,ω-alkylene group with up to four carbons; and
wherein R² is hydrogen or saturated or unsaturated alkyl with up to 6 carbons; or alkoxy with up to 3 carbons; or trialkylsilyl (trimethylsilyl, triethylsilyl, tert-butyldimethylsilyl, dimethylphenylsilyl); or rialkylsilyloxy (trimethylsilyloxy, tert-butyldimethylsilyloxy, triethylsilyloxy, dimethylphenylsilyloxy); and
wherein R³ is hydrogen or saturated or unsaturated alkyl with up to 6 carbons; or alkoxy with up to 3 carbons; or trialkylsilyl (trimethylsilyl, triethylsilyl, tert-butyldimethylsilyl, dimethylphenylsilyl); or trialkylsilyloxy (trimethylsilyloxy, tert-butyldimethylsilyloxy, triethylsilyloxy, dimethylphenylsilyloxy); and
wherein R⁵ is hydrogen or trialkylsilyl (trimethylsilyl, triethylsilyl, tert-butyldimethylsilyl, dimethylphenylsilyl); and
wherein X is a heteroatomic group (alcohol, ester, amine, amide, sulfonamide, halide, sulfonate ester).

4. A compound comprising the following structure: 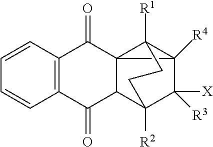
wherein R¹ is hydrogen or saturated or unsaturated alkyl with up to 6 carbons; or alkoxy with up to 3 carbons; or trialkylsilyl (trimethylsilyl, triethylsilyl, tert-butyldimethylsilyl, dimethylphenylsilyl); or trialkylsilyloxy (trimethylsilyloxy, tert-butyldimethylsilyloxy, triethylsilyloxy, dimethylphenylsilyloxy); and
wherein R² is hydrogen or saturated or unsaturated alkyl with up to 6 carbons; or alkoxy with up to 3 carbons; or trialkylsilyl (trimethylsilyl, triethylsilyl, tert-butyldimethylsilyl, dimethylphenylsilyl); or rialkylsilyloxy (trimethylsilyloxy, tert-butyldimethylsilyloxy, triethylsilyloxy, dimethylphenylsilyloxy); and
wherein R³ is hydrogen or saturated or unsaturated alkyl with up to 6 carbons; or alkoxy with up to 3 carbons; or trialkylsilyl (trimethylsilyl, triethylsilyl, tert-butyldimethylsilyl, dimethylphenylsilyl); or trialkylsilyloxy (trimethylsilyloxy, tert-butyldimethylsilyloxy, triethylsilyloxy, dimethylphenylsilyloxy); and
wherein R⁴ hydrogen or saturated or unsaturated alkyl with up to 6 carbons; or alkoxy with up to 3 carbons; or trialkylsilyl (trimethylsilyl, triethylsilyl, tert-butyldimethylsilyl, dimethylphenylsilyl); or trialkylsilyloxy (trimethylsilyloxy, tert-butyldimethylsilyloxy, triethylsilyloxy, dimethylphenylsilyloxy); and
wherein X is a heteroatomic group (alcohol, ester, amine, amide, sulfonamide, halide, sulfonate ester);
or
wherein R¹ and R² comprise a substituted or unsubstituted, saturated or unsaturated α,ω-alkylene group with up to four carbons; and
wherein R³ is hydrogen or saturated or unsaturated alkyl with up to 6 carbons; or alkoxy with up to 3 carbons; or trialkylsilyl (trimethylsilyl, triethylsilyl, tert-butyldimethylsilyl, dimethylphenylsilyl); or rialkylsilyloxy (trimethylsilyloxy, tert-butyldimethylsilyloxy, triethylsilyloxy, dimethylphenylsilyloxy); and
wherein R⁴ is hydrogen or saturated or unsaturated alkyl with up to 6 carbons; or alkoxy with up to 3 carbons; or trialkylsilyl (trimethylsilyl, triethylsilyl, tert-butyldimethylsilyl, dimethylphenylsilyl); or trialkylsilyloxy (trimethylsilyloxy, tert-butyldimethylsilyloxy, triethylsilyloxy, dimethylphenylsilyloxy); and
wherein X is a heteroatomic group (alcohol, ester, amine, amide, sulfonamide, halide, sulfonate ester);
or
wherein R¹ and R⁴ comprise a substituted or unsubstituted, saturated or unsaturated α,ω-alkylene group with up to four carbons; and
wherein R² is hydrogen or saturated or unsaturated alkyl with up to 6 carbons; or alkoxy with up to 3 carbons; or trialkylsilyl (trimethylsilyl, triethylsilyl, tert-butyldimethylsilyl, dimethylphenylsilyl); or rialkylsilyloxy (trimethylsilyloxy, tert-butyldimethylsilyloxy, triethylsilyloxy, dimethylphenylsilyloxy); and
wherein R³ is hydrogen or saturated or unsaturated alkyl with up to 6 carbons; or alkoxy with up to 3 carbons; or trialkylsilyl (trimethylsilyl, triethylsilyl, tert-butyldimethylsilyl, dimethylphenylsilyl); or trialkylsilyloxy (trimethylsilyloxy, tert-butyldimethylsilyloxy, triethylsilyloxy, dimethylphenylsilyloxy); and
wherein X is a heteroatomic group (alcohol, ester, amine, amide, sulfonamide, halide, sulfonate ester).

5. A method for enhancing the effectiveness of an anti-coagulant composition comprising the step of administering the anticoagulant composition in conjunction with an effective amount of a composition comprising the following structure: 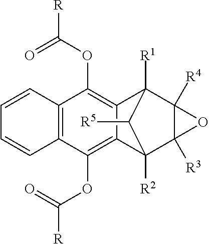
wherein R is hydrogen or saturated or unsaturated alkyl with up to 6 carbons; and
wherein R¹ is hydrogen or saturated or unsaturated alkyl with up to 6 carbons; or alkoxy with up to 3 carbons; or trialkylsilyl (trimethylsilyl, triethylsilyl, tert-butyldimethylsilyl, dimethylphenylsilyl); or trialkylsilyloxy (trimethylsilyloxy, tert-butyldimethylsilyloxy, triethylsilyloxy, dimethylphenylsilyloxy); and
wherein R² is hydrogen or saturated or unsaturated alkyl with up to 6 carbons; or alkoxy with up to 3 carbons; or trialkylsilyl (trimethylsilyl, triethylsilyl, tert-butyldimethylsilyl, dimethylphenylsilyl); or rialkylsilyloxy (trimethylsilyloxy, tert-butyldimethylsilyloxy, triethylsilyloxy, dimethylphenylsilyloxy); and
R³ is hydrogen or saturated or unsaturated alkyl with up to 6 carbons; or alkoxy with up to 3 carbons; or trialkylsilyl (trimethylsilyl, triethylsilyl, tert-butyldimethylsilyl, dimethylphenylsilyl); or trialkylsilyloxy (trimethylsilyloxy, tert-butyldimethylsilyloxy, triethylsilyloxy, dimethylphenylsilyloxy); and
R⁴ hydrogen or saturated or unsaturated alkyl with up to 6 carbons; or alkoxy with up to 3 carbons; or trialkylsilyl (trimethylsilyl, triethylsilyl, tert-butyldimethylsilyl, dimethylphenylsilyl); or trialkylsilyloxy (trimethylsilyloxy, tert-butyldimethylsilyloxy, triethylsilyloxy, dimethylphenylsilyloxy); and
R⁵ is hydrogen or trialkylsilyl (trimethylsilyl, triethylsilyl, tert-butyldimethylsilyl, dimethylphenylsilyl);
or
wherein R¹ and R² comprise a substituted or unsubstituted, saturated or unsaturated α,ω-alkylene group with up to four carbons; and
wherein R³ is hydrogen or saturated or unsaturated alkyl with up to 6 carbons; or alkoxy with up to 3 carbons; or trialkylsilyl (trimethylsilyl, triethylsilyl, tert-butyldimethylsilyl, dimethylphenylsilyl); or rialkylsilyloxy (trimethylsilyloxy, tert-butyldimethylsilyloxy, triethylsilyloxy, dimethylphenylsilyloxy); and
wherein R⁴ is hydrogen or saturated or unsaturated alkyl with up to 6 carbons; or alkoxy with up to 3 carbons; or trialkylsilyl (trimethylsilyl, triethylsilyl, tert-butyldimethylsilyl, dimethylphenylsilyl); or trialkylsilyloxy (trimethylsilyloxy, tert-butyldimethylsilyloxy, triethylsilyloxy, dimethylphenylsilyloxy); and
wherein R⁵ is hydrogen or trialkylsilyl (trimethylsilyl, triethylsilyl, tert-butyldimethylsilyl, dimethylphenylsilyl);
or
wherein R¹ and R⁴ comprise a substituted or unsubstituted, saturated or unsaturated α,ω-alkylene group with up to four carbons; and
wherein R² is hydrogen or saturated or unsaturated alkyl with up to 6 carbons; or alkoxy with up to 3 carbons; or trialkylsilyl (trimethylsilyl, triethylsilyl, tert-butyldimethylsilyl, dimethylphenylsilyl); or rialkylsilyloxy (trimethylsilyloxy, tert-butyldimethylsilyloxy, triethylsilyloxy, dimethylphenylsilyloxy); and
wherein R³ is hydrogen or saturated or unsaturated alkyl with up to 6 carbons; or alkoxy with up to 3 carbons; or trialkylsilyl (trimethylsilyl, triethylsilyl, tert-butyldimethylsilyl, dimethylphenylsilyl); or trialkylsilyloxy (trimethylsilyloxy, tert-butyldimethylsilyloxy, triethylsilyloxy, dimethylphenylsilyloxy); and
wherein R⁵ is hydrogen or trialkylsilyl (trimethylsilyl, triethylsilyl, tert-butyldimethylsilyl, dimethylphenylsilyl);
and
wherein the anticoagulant composition is a coumarin-based anticoagulant or metabolite thereof with the general structure 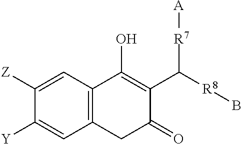
wherein R⁷ is a substituted alkyl group with up to 6 carbons; and
A is a group chosen from an alcohol, an ester, an ether, an ester, or a ketone; and
wherein R⁸ is a substituted alkyl group with up to 6 carbons; and
B is a an aryl group (phenyl, biphenyl, furyl, 4′-bromo-4,1′-biphenyl);
or
wherein R⁷ and R⁸ constitute a cycloalkyl group with up to 7 carbons; and
A is a group chosen from an alcohol, an ester, an ether, an ester, or a ketone; and
B is a an aryl group (phenyl, biphenyl, furyl, 4′-bromo-4,1′-biphenyl); and
wherein X is hydrogen or hydroxy; and
wherein Y is hydrogen or hydroxy.

6. A method for enhancing the effectiveness of an anti-coagulant composition comprising the step of administering the anticoagulant composition in conjunction with an effective amount of a composition comprising the following structure: 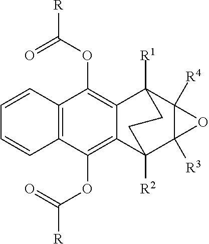
wherein R is hydrogen or saturated or unsaturated alkyl with up to 6 carbons; and
wherein R¹ is hydrogen or saturated or unsaturated alkyl with up to 6 carbons; or alkoxy with up to 3 carbons; or trialkylsilyl (trimethylsilyl, triethylsilyl, tert-butyldimethylsilyl, dimethylphenylsilyl); or trialkylsilyloxy (trimethylsilyloxy, tert-butyldimethylsilyloxy, triethylsilyloxy, dimethylphenylsilyloxy); and
wherein R² is hydrogen or saturated or unsaturated alkyl with up to 6 carbons; or alkoxy with up to 3 carbons; or trialkylsilyl (trimethylsilyl, triethylsilyl, tert-butyldimethylsilyl, dimethylphenylsilyl); or rialkylsilyloxy (trimethylsilyloxy, tert-butyldimethylsilyloxy, triethylsilyloxy, dimethylphenylsilyloxy); and
R³ is hydrogen or saturated or unsaturated alkyl with up to 6 carbons; or alkoxy with up to 3 carbons; or trialkylsilyl (trimethylsilyl, triethylsilyl, tert-butyldimethylsilyl, dimethylphenylsilyl); or trialkylsilyloxy (trimethylsilyloxy, tert-butyldimethylsilyloxy, triethylsilyloxy, dimethylphenylsilyloxy); and
R⁴ hydrogen or saturated or unsaturated alkyl with up to 6 carbons; or alkoxy with up to 3 carbons; or trialkylsilyl (trimethylsilyl, triethylsilyl, tert-butyldimethylsilyl, dimethylphenylsilyl); or trialkylsilyloxy (trimethylsilyloxy, tert-butyldimethylsilyloxy, triethylsilyloxy, dimethylphenylsilyloxy);
or
wherein R¹ and R² comprise a substituted or unsubstituted, saturated or unsaturated α,ω-alkylene group with up to four carbons; and
wherein R³ is hydrogen or saturated or unsaturated alkyl with up to 6 carbons; or alkoxy with up to 3 carbons; or trialkylsilyl (trimethylsilyl, triethylsilyl, tert-butyldimethylsilyl, dimethylphenylsilyl); or rialkylsilyloxy (trimethylsilyloxy, tert-butyldimethylsilyloxy, triethylsilyloxy, dimethylphenylsilyloxy); and
wherein R⁴ is hydrogen or saturated or unsaturated alkyl with up to 6 carbons; or alkoxy with up to 3 carbons; or trialkylsilyl (trimethylsilyl, triethylsilyl, tert-butyldimethylsilyl, dimethylphenylsilyl); or trialkylsilyloxy (trimethylsilyloxy, tert-butyldimethylsilyloxy, triethylsilyloxy, dimethylphenylsilyloxy);
or
wherein R¹ and R⁴ comprise a substituted or unsubstituted, saturated or unsaturated α,ω-alkylene group with up to four carbons; and
wherein R² is hydrogen or saturated or unsaturated alkyl with up to 6 carbons; or alkoxy with up to 3 carbons; or trialkylsilyl (trimethylsilyl, triethylsilyl, tert-butyldimethylsilyl, dimethylphenylsilyl); or rialkylsilyloxy (trimethylsilyloxy, tert-butyldimethylsilyloxy, triethylsilyloxy, dimethylphenylsilyloxy); and
wherein R³ is hydrogen or saturated or unsaturated alkyl with up to 6 carbons; or alkoxy with up to 3 carbons; or trialkylsilyl (trimethylsilyl, triethylsilyl, tert-butyl dimethylsilyl, dim ethyl phenyl silyl); or trialkylsilyloxy (trimethylsilyloxy, tert-butyl dimethylsilyloxy, triethylsilyloxy, dim ethyl phenylsilyloxy);
and
wherein the anticoagulant composition is a coumarin-based anticoagulant or metabolite thereof with the general structure 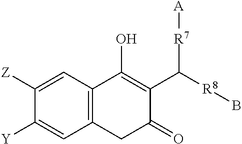
wherein R⁷ is a substituted alkyl group with up to 6 carbons; and
A is a group chosen from an alcohol, an ester, an ether, an ester, or a ketone; and
wherein R⁸ is a substituted alkyl group with up to 6 carbons; and
B is a an aryl group (phenyl, biphenyl, furyl, 4′-bromo-4,1′-biphenyl);
or
wherein R⁷ and R⁸ constitute a cycloalkyl group with up to 7 carbons; and
A is a group chosen from an alcohol, an ester, an ether, an ester, or a ketone; and
B is a an aryl group (phenyl, biphenyl, furyl, 4′-bromo-4,1′-biphenyl); and
wherein X is hydrogen or hydroxy; and
wherein Y is hydrogen or hydroxy.

7. A method for enhancing the effectiveness of an anti-coagulant composition comprising the step of administering the anticoagulant composition in conjunction with an effective amount of a composition comprising the following structure: 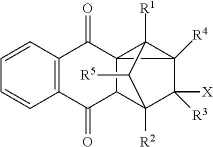
wherein R¹ is hydrogen or saturated or unsaturated alkyl with up to 6 carbons; or alkoxy with up to 3 carbons; or trialkylsilyl (trimethylsilyl, triethylsilyl, tert-butyldimethylsilyl, dimethylphenylsilyl); or trialkylsilyloxy (trimethylsilyloxy, tert-butyldimethylsilyloxy, triethylsilyloxy, dimethylphenylsilyloxy); and
wherein R² is hydrogen or saturated or unsaturated alkyl with up to 6 carbons; or alkoxy with up to 3 carbons; or trialkylsilyl (trimethylsilyl, triethylsilyl, tert-butyldimethylsilyl, dimethylphenylsilyl); or rialkylsilyloxy (trimethylsilyloxy, tert-butyldimethylsilyloxy, triethylsilyloxy, dimethylphenylsilyloxy); and
wherein R³ is hydrogen or saturated or unsaturated alkyl with up to 6 carbons; or alkoxy with up to 3 carbons; or trialkylsilyl (trimethylsilyl, triethylsilyl, tert-butyldimethylsilyl, dimethylphenylsilyl); or trialkylsilyloxy (trimethylsilyloxy, tert-butyldimethylsilyloxy, triethylsilyloxy, dimethylphenylsilyloxy); and
wherein R⁴ hydrogen or saturated or unsaturated alkyl with up to 6 carbons; or alkoxy with up to 3 carbons; or trialkylsilyl (trimethylsilyl, triethylsilyl, tert-butyldimethylsilyl, dimethylphenylsilyl); or trialkylsilyloxy (trimethylsilyloxy, tert-butyldimethylsilyloxy, triethylsilyloxy, dimethylphenylsilyloxy); and
wherein R⁵ is hydrogen or trialkylsilyl (trimethylsilyl, triethylsilyl, tert-butyldimethylsilyl, dimethylphenylsilyl); and
wherein X is a heteroatomic group (alcohol, ester, amine, amide, sulfonamide, halide, sulfonate ester);
or
wherein R¹ and R² comprise a substituted or unsubstituted, saturated or unsaturated α,ω-alkylene group with up to four carbons; and
wherein R³ is hydrogen or saturated or unsaturated alkyl with up to 6 carbons; or alkoxy with up to 3 carbons; or trialkylsilyl (trimethylsilyl, triethylsilyl, tert-butyldimethylsilyl, dimethylphenylsilyl); or rialkylsilyloxy (trimethylsilyloxy, tert-butyldimethylsilyloxy, triethylsilyloxy, dimethylphenylsilyloxy); and
wherein R⁴ is hydrogen or saturated or unsaturated alkyl with up to 6 carbons; or alkoxy with up to 3 carbons; or trialkylsilyl (trimethylsilyl, triethylsilyl, tert-butyldimethylsilyl, dimethylphenylsilyl); or trialkylsilyloxy (trimethylsilyloxy, tert-butyldimethylsilyloxy, triethylsilyloxy, dimethylphenylsilyloxy); and
wherein R⁵ is hydrogen or trialkylsilyl (trimethylsilyl, triethylsilyl, tert-butyldimethylsilyl, dimethylphenylsilyl); and
wherein X is a heteroatomic group (alcohol, ester, amine, amide, sulfonamide, halide, sulfonate ester);
or
wherein R¹ and R⁴ comprise a substituted or unsubstituted, saturated or unsaturated α,ω-alkylene group with up to four carbons; and
wherein R² is hydrogen or saturated or unsaturated alkyl with up to 6 carbons; or alkoxy with up to 3 carbons; or trialkylsilyl (trimethylsilyl, triethylsilyl, tert-butyldimethylsilyl, dimethylphenylsilyl); or rialkylsilyloxy (trimethylsilyloxy, tert-butyldimethylsilyloxy, triethylsilyloxy, dimethylphenylsilyloxy); and
wherein R³ is hydrogen or saturated or unsaturated alkyl with up to 6 carbons; or alkoxy with up to 3 carbons; or trialkylsilyl (trimethylsilyl, triethylsilyl, tert-butyldimethylsilyl, dimethylphenylsilyl); or trialkylsilyloxy (trimethylsilyloxy, tert-butyldimethylsilyloxy, triethylsilyloxy, dimethylphenylsilyloxy); and
wherein R⁵ is hydrogen or trialkylsilyl (trimethylsilyl, triethylsilyl, tert-butyldimethylsilyl, dimethylphenylsilyl); and
wherein X is a heteroatomic group (alcohol, ester, amine, amide, sulfonamide, halide, sulfonate ester);
and
wherein the anticoagulant composition is a coumarin-based anticoagulant or metabolite thereof with the general structure 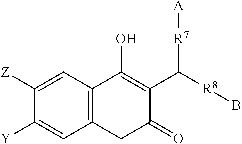
wherein R⁷ is a substituted alkyl group with up to 6 carbons; and
A is a group chosen from an alcohol, an ester, an ether, an ester, or a ketone; and
wherein R⁸ is a substituted alkyl group with up to 6 carbons; and
B is a an aryl group (phenyl, biphenyl, furyl, 4′-bromo-4,1′-biphenyl);
or
wherein R⁷ and R⁸ constitute a cycloalkyl group with up to 7 carbons; and
A is a group chosen from an alcohol, an ester, an ether, an ester, or a ketone; and
B is a an aryl group (phenyl, biphenyl, furyl, 4′-bromo-4,1′-biphenyl); and
wherein X is hydrogen or hydroxy; and
wherein Y is hydrogen or hydroxy.

8. A method for enhancing the effectiveness of an anti-coagulant composition comprising the step of administering the anticoagulant composition in conjunction with an effective amount of a composition comprising the following structure: 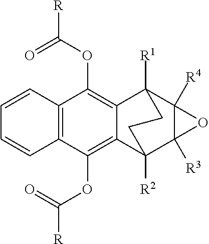
wherein R¹ is hydrogen or saturated or unsaturated alkyl with up to 6 carbons; or alkoxy with up to 3 carbons; or trialkylsilyl (trimethylsilyl, triethylsilyl, tert-butyldimethylsilyl, dimethylphenylsilyl); or trialkylsilyloxy (trimethylsilyloxy, tert-butyldimethylsilyloxy, triethylsilyloxy, dimethylphenylsilyloxy); and
wherein R² is hydrogen or saturated or unsaturated alkyl with up to 6 carbons; or alkoxy with up to 3 carbons; or trialkylsilyl (trimethylsilyl, triethylsilyl, tert-butyldimethylsilyl, dimethylphenylsilyl); or rialkylsilyloxy (trimethylsilyloxy, tert-butyldimethylsilyloxy, triethylsilyloxy, dimethylphenylsilyloxy); and
wherein R³ is hydrogen or saturated or unsaturated alkyl with up to 6 carbons; or alkoxy with up to 3 carbons; or trialkylsilyl (trimethylsilyl, triethylsilyl, tert-butyldimethylsilyl, dimethylphenylsilyl); or trialkylsilyloxy (trimethylsilyloxy, tert-butyldimethylsilyloxy, triethylsilyloxy, dimethylphenylsilyloxy); and
wherein R⁴ hydrogen or saturated or unsaturated alkyl with up to 6 carbons; or alkoxy with up to 3 carbons; or trialkylsilyl (trimethylsilyl, triethylsilyl, tert-butyldimethylsilyl, dimethylphenylsilyl); or trialkylsilyloxy (trimethylsilyloxy, tert-butyldimethylsilyloxy, triethylsilyloxy, dimethylphenylsilyloxy); and
wherein X is a heteroatomic group (alcohol, ester, amine, amide, sulfonamide, halide, sulfonate ester);
or
wherein R¹ and R² comprise a substituted or unsubstituted, saturated or unsaturated α,ω-alkylene group with up to four carbons; and
wherein R³ is hydrogen or saturated or unsaturated alkyl with up to 6 carbons; or alkoxy with up to 3 carbons; or trialkylsilyl (trimethylsilyl, triethylsilyl, tert-butyldimethylsilyl, dimethylphenylsilyl); or rialkylsilyloxy (trimethylsilyloxy, tert-butyldimethylsilyloxy, triethylsilyloxy, dimethylphenylsilyloxy); and
wherein R⁴ is hydrogen or saturated or unsaturated alkyl with up to 6 carbons; or alkoxy with up to 3 carbons; or trialkylsilyl (trimethylsilyl, triethylsilyl, tert-butyldimethylsilyl, dimethylphenylsilyl); or trialkylsilyloxy (trimethylsilyloxy, tert-butyldimethylsilyloxy, triethylsilyloxy, dimethylphenylsilyloxy); and
wherein X is a heteroatomic group (alcohol, ester, amine, amide, sulfonamide, halide, sulfonate ester);
or
wherein R¹ and R⁴ comprise a substituted or unsubstituted, saturated or unsaturated α,ω-alkylene group with up to four carbons; and
wherein R² is hydrogen or saturated or unsaturated alkyl with up to 6 carbons; or alkoxy with up to 3 carbons; or trialkylsilyl (trimethylsilyl, triethylsilyl, tert-butyldimethylsilyl, dimethylphenylsilyl); or rialkylsilyloxy (trimethylsilyloxy, tert-butyldimethylsilyloxy, triethylsilyloxy, dimethylphenylsilyloxy); and
wherein R³ is hydrogen or saturated or unsaturated alkyl with up to 6 carbons; or alkoxy with up to 3 carbons; or trialkylsilyl (trimethylsilyl, triethylsilyl, tert-butyldimethylsilyl, dimethylphenylsilyl); or trialkylsilyloxy (trimethylsilyloxy, tert-butyldimethylsilyloxy, triethylsilyloxy, dimethylphenylsilyloxy); and
wherein X is a heteroatomic group (alcohol, ester, amine, amide, sulfonamide, halide, sulfonate ester);
and
wherein the anticoagulant composition is a coumarin-based anticoagulant or metabolite thereof with the general structure 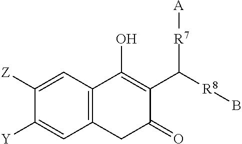
wherein R⁷ is a substituted alkyl group with up to 6 carbons; and
A is a group chosen from an alcohol, an ester, an ether, an ester, or a ketone; and
wherein R⁸ is a substituted alkyl group with up to 6 carbons; and
B is a an aryl group (phenyl, biphenyl, furyl, 4′-bromo-4,1′-biphenyl);
or
wherein R⁷ and R⁸ constitute a cycloalkyl group with up to 7 carbons; and
A is a group chosen from an alcohol, an ester, an ether, an ester, or a ketone; and
B is a an aryl group (phenyl, biphenyl, furyl, 4′-bromo-4,1′-biphenyl); and
wherein X is hydrogen or hydroxy; and
wherein Y is hydrogen or hydroxy.

9. The composition in claim 5, wherein the anticoagulant is a coumarin-based anticoagulant.

10. The composition in claim 6, wherein the anticoagulant is a coumarin-based anticoagulant.

11. The composition in claim 7, wherein the anticoagulant is a coumarin-based anticoagulant.

12. The composition in claim 8, wherein the anticoagulant is a coumarin-based anticoagulant.

13. The composition in claim 5, wherein the anticoagulant is warfarin.

14. The composition in claim 6, wherein the anticoagulant is warfarin.

15. The composition in claim 7, wherein the anticoagulant is warfarin.

16. The composition in claim 8, wherein the anticoagulant is warfarin.

17. The composition in claim 5, wherein the anticoagulant is a second-generation coumarin anticoagulant.

18. The composition in claim 6, wherein the anticoagulant is a second-generation coumarin anticoagulant.

19. The composition in claim 7, wherein the anticoagulant is a second-generation coumarin anticoagulant.

20. The composition in claim 8, wherein the anticoagulant is a second-generation coumarin anticoagulant.

21. The composition in claim 7, wherein the adjuvant composition is obtained by hydrolysis of the composition in claim 5 and wherein the anticoagulant is a coumarin-based anticoagulant.

22. The composition in claim 8, wherein the adjuvant composition is obtained by hydrolysis of the composition in claim 6 and wherein the anticoagulant is a coumarin-based anticoagulant.

23. The composition in claim 7, wherein the adjuvant composition is obtained by hydrolysis of the composition in claim 5 and wherein the anticoagulant is warfarin.

24. The composition in claim 8, wherein the adjuvant composition is obtained by hydrolysis of the composition in claim 6 and wherein the anticoagulant is warfarin.

25. The composition in claim 7, wherein the adjuvant composition is obtained by hydrolysis of the composition in claim 5 and wherein the anticoagulant is a second-generation coumarin anticoagulant.

26. The composition in claim 8, wherein the adjuvant composition is obtained by hydrolysis of the composition in claim 6 and wherein the anticoagulant is a second-generation coumarin anticoagulant.
